(19) United States
(12) Patent Application Publication
(10) Pub. No.: US 2018/0038930 A1
(43) Pub. Date: Feb. 8, 2018

(54) METHOD AND APPARATUS FOR SETTING AND/OR ADJUSTING A PARAMETER VALUE OF A PARAMETER OF A MAGNETIC RESONANCE PROTOCOL FOR A MAGNETIC RESONANCE SEQUENCE

(71) Applicant: Siemens Healthcare GmbH, Erlangen (DE)

(72) Inventor: Maria Kroell, Erlangen (DE)

(73) Assignee: Siemens Healthcare GmbH, Erlangen (DE)

(21) Appl. No.: 15/670,029

(22) Filed: Aug. 7, 2017

(30) Foreign Application Priority Data
Aug. 8, 2016 (EP) 16183218.3

(51) Int. Cl. G01R 33/54; A61B 5/055; G06N 99/00

(52) U.S. Cl. CPC G01R 33/543; G01R 33/546; G06N 99/005; A61B 5/0555

(57) ABSTRACT

In a method and apparatus for setting and/or adjusting a parameter value of at least one parameter of a magnetic resonance protocol for at least one magnetic resonance sequence, a manual entry that selects the magnetic resonance protocol is made into a computer, and a further entry is made that provides boundary conditions for setting and/or adjusting the parameter value of the at least one parameter of the magnetic resonance protocol. The computer provides setting criteria from previous settings and/or adjustments of the parameter value of the at least one parameter of the magnetic resonance protocol. The computer sets and/or adjusts the parameter value of the at least one parameter of the magnetic resonance protocol on the basis of the provided boundary conditions and on the basis of the provided setting criteria.

### BACKGROUND OF THE INVENTION

### Field of the Invention

The present invention relates to a method and a magnetic resonance apparatus for setting and/or adjusting a parameter value of at least one parameter of a magnetic resonance protocol for at least one magnetic resonance sequence. The parameter value of the at least one parameter of the magnetic resonance protocol is set and/or adjusted on the basis of provided boundary conditions and on the basis of provided setting criteria.

### Description of the Prior Art

In order to produce magnetic resonance images of a patient, medical operating personnel must first select magnetic resonance protocols. These magnetic resonance protocols have a multiplicity of parameters, of which individual parameters or even a number of parameters must be set and/or adjusted by the medical operating personnel. The large number of parameters that can be set and/or adjusted means that it is often difficult for the medical operating personnel, in particular for medical operating personnel who are inexperienced in setting and/or adjusting parameters, to make a correct parameter selection and/or to set a correct parameter value for a successful magnetic resonance examination. Moreover, it is often difficult for a user to identify which parameters within a magnetic resonance sequence are correlated with one another, with the result that a change in one parameter inevitably causes a change in other parameters. This correlation between individual parameters also makes it harder for a user to set and/or adjust the parameter values within a magnetic resonance protocol.

### SUMMARY OF THE INVENTION

An object of the present invention is to make it possible for a user to set and/or adjust parameter values of a magnetic resonance protocol of a magnetic resonance sequence easily.

A method in accordance with the invention for setting and/or adjusting a parameter value of at least one parameter of a magnetic resonance protocol for at least one magnetic resonance sequence has the following steps:

An entry is made into a computer that selects a magnetic resonance protocol.

Another entry is made into the computer that designates boundary conditions for setting and/or adjusting the parameter value of the at least one parameter of the magnetic resonance protocol.

Setting criteria from previous settings and/or adjustments of the parameter value of the at least one parameter of the magnetic resonance protocol are entered into or accessed by the computer.

The parameter value of the at least one parameter of the magnetic resonance protocol is set and/or adjusted by the computer on the basis of the provided boundary conditions and on the basis of the provided setting criteria. The set or adjusted parameter value is then made available from the computer in electronic form, or the magnetic resonance protocol with the set or adjusted parameter therein is made available from the computer in electronic form.

In this context, a parameter shall be understood to mean a protocol-specific parameter that preferably can be set and/or adjusted by a user before a magnetic resonance examination on a patient. The protocol-specific parameter may include, for example, an echo time and/or a slice thickness and/or a slice orientation, etc. For instance, a user can influence an image quality by skilled selection and/or adjustment of the individual parameters of a magnetic resonance protocol.

A magnetic resonance sequence shall be understood to mean a defined sequence of separate gradient pulses and/or RF pulses. For the magnetic resonance sequence, the magnetic resonance protocol typically contains parameters defining the individual criteria for the execution of the magnetic resonance sequence.

The magnetic resonance protocol can be selected in this case by a user, for instance by a doctor and/or a radiographer. The magnetic resonance protocol is preferably selected via means of a user interface of the computer, which can be used by the user to make his or her selection. This user interface can be a component of the magnetic resonance apparatus or can be a mobile user interface such as a mobile touchscreen and/or tablet PC, for example, which is in communication with a control computer and/or a server that allow access to the magnetic resonance protocols. It can also be possible for the magnetic resonance protocol to be selected automatically and/or autonomously by the computer on the basis of provided patient information, in particular by the control computer of the magnetic resonance apparatus.

The provision of boundary conditions for setting and/or adjusting the parameter value is preferably performed by the user. The boundary conditions can be target-oriented boundary conditions such as a measurement time and/or an image quality and/or a slice thickness and/or a contrast ratio and/or signal/noise ratio, etc. To meet the boundary conditions, in particular the target-oriented boundary conditions, it is usually necessary to adjust and/or set individual parameters. In addition, it is also conceivable for the boundary conditions to include a value range for the parameter value of the at least one parameter. The user provides the boundary conditions via the user interface.

The boundary conditions may also include fixed values of parameters, which must not be changed in order for the target-oriented boundary conditions to be met. In this case, the fixed values of individual parameters may already be stored and/or saved inside the magnetic resonance protocol. In addition, it is also possible for a user to use the user interface to specify individual parameters having fixed parameter values. The setting criteria preferably are embodied as information that is stored in a storage medium and/or a database and can be retrieved from the storage medium and/or the database in the provision step. The setting criteria can be information on, and/or settings of, parameter values of magnetic resonance sequences already performed and/or used in the past. The setting criteria can also include evaluation information on the set parameter values, such evaluation information having been saved by a user. The provision of setting criteria is preferably performed by the control computer, which can access the storage medium and/or the database for this purpose.

The method steps of providing boundary conditions for setting and/or adjusting the parameter value and of providing setting criteria can be performed also simultaneously in time or in any sequence in time.

The method step of setting and/or adjusting the parameter value of the at least one parameter of the magnetic resonance protocol on the basis of the provided boundary conditions and on the basis of the provided setting criteria is preferably performed automatically and/or autonomously by the control computer and/or a processor. The control computer and/or the processor have the necessary computer programs and/or necessary software for implementing the method.

The control computer and/or processor can have a central control computer and/or a central processor, which have available stored setting criteria of magnetic resonance protocols that are performed on different magnetic resonance apparatuses. Alternatively or additionally, the control computer can be a component of the magnetic resonance apparatus, so that only the stored setting criteria that have been acquired by the operation of this magnetic resonance apparatus can be used.

The method according to the invention can be used to achieve automated setting and/or adjustment of the parameter values for a magnetic resonance protocol and hence to provide simple and time-saving adjustment and/or setting of parameters for a user. All that is required of the user for setting and/or adjusting the parameter values is just to select the magnetic resonance sequence and to specify and/or provide at least some of the boundary conditions. In particular, this allows new and/or inexperienced users to use individual magnetic resonance sequences optimally, with the result that the acquired magnetic resonance data can be of a higher quality. In addition, for the purpose of setting and/or adjusting the parameter value of the at least one parameter, all previously used and available setting criteria can be used automatically, thereby also reducing a possibility of mistakes when setting and/or adjusting the parameter values of the magnetic resonance protocol. This can also ensure that the acquired image data are of a high quality.

The parameter value of the at least one parameter of the magnetic resonance protocol is set and/or adjusted automatically and/or autonomously by the control computer. The control computer can execute a self-learning algorithm, which adjusts and/or sets the parameter values on the basis of the setting criteria and/or boundary conditions. In this case, an optimum setting and/or adjustment, in particular a custom and/or situation-specific setting and/or adjustment, of the parameter values for individual parameters can be made automatically according to the boundary conditions. Moreover, the self-learning algorithm can be used to prevent parameter values repeatedly being set and/or adjusted incorrectly. In addition, a setting and/or an adjustment of the protocol parameters of the parameters of the magnetic resonance protocol can be constantly improved, such as with every setting and/or adjustment and subsequent evaluation of the results.

The self-learning algorithm is typically based on machine learning, which involves generating knowledge from experience. Machine learning is performed by artificial neural networks. The self-learning algorithm can use machine learning to recognize patterns and regularities in training data, in particular in the saved set and/or adjusted parameter values and the evaluation thereof. In this context, the self-learning algorithm can learn from examples and generalize these at the end of the training phase.

The self-learning algorithm and/or machine learning can be based on a deep-learning method, in which knowledge is generated from experience. With the deep-learning method, artificial neural networks are arranged in layers, which use ever more complex features to recognize, for example, the content of image data and/or contrasts in image data. This can used, for example, to categorize large amounts of data.

In accordance with the invention, the self-learning algorithm, in particular machine learning such as the deep-learning method, for instance, takes into account all the setting criteria stored in the storage medium and/or the database for adjusting and/or setting the parameter value of the at least one parameter of the magnetic resonance protocol. This can achieve an optimum adjustment and/or setting of the parameter value, because statistical and/or random errors in the adjustment and/or setting of the parameter value can be reduced and/or eliminated thanks to the large amount of data.

In an embodiment of the invention, the boundary conditions for setting and/or adjusting the parameter value of the at least one parameter of the magnetic resonance protocol include a value range for the parameter value to be set and/or adjusted. A value range shall be understood to mean a range that contains those values that define a setting range and/or adjustment range for the parameter value of the at least one parameter. This value range for the parameter value can be defined manually by a user. Alternatively, the value range for the parameter value can be stored in a database, such as when the value range for the parameter value is always meant to involve a constant range.

A practical value range can be defined in this way. In addition, personal specifications of a user can be taken into account in adjusting and/or setting the parameter value of the at least one parameter. Furthermore, examination specifications, for instance in a hospital, in which specifications the value range is always of constant size, can be taken into account in such a way.

In another embodiment, the boundary conditions for setting and/or adjusting the parameter value of the at least one parameter of the magnetic resonance protocol include at least one additional parameter value of an additional parameter of the magnetic resonance protocol, wherein the additional parameter value involves a fixed parameter value. A fixed parameter value shall be understood to mean in this context a parameter value of a parameter of a magnetic resonance protocol, this parameter value having a fixed value regardless of adjustments and/or changes to additional parameter values, in particular when there is a change and/or adjustment to the parameter value to be set. In order to meet the boundary conditions, setting and/or adjusting the parameter value often involves changes to additional parameter values that are correlated with the parameter value to be set and/or adjusted. In contrast, the fixed parameter value does not change its value, regardless of the boundary conditions and/or the setting and/or adjusting of the parameter value. The user can define and/or specify a parameter value as a fixed parameter value. A user can thereby define and/or specify individual parameter values of parameters that are meant to always involve a constant value for every measurement.

In another embodiment of the invention, the boundary conditions for setting and/or adjusting the parameter value of the at least one parameter of the magnetic resonance protocol include target-oriented boundary conditions. Target-oriented boundary conditions shall be understood to mean boundary conditions that include a target and/or result of the magnetic resonance examination. The target-oriented boundary conditions can be a measurement time of the magnetic resonance sequence and/or an image quality of the acquired image data and/or a slice thickness and/or a contrast ratio and/or signal/noise ratio, etc. This means that it is simply required for a user to specify only the target-oriented and/or objective-oriented boundary conditions, such as a T1 contrast measurement having a specific slice thickness and a defined image coverage for instance, and a subsequent adjustment and/or setting of the parameter values of all parameters to be adjusted and/or set is performed automatically and/or autonomously by the self-learning algorithm, by machine learning such as the deep-learning method, for instance.

The boundary conditions for setting and/or adjusting the parameter value of the at least one parameter of the magnetic resonance protocol are preferably specific to a user and/or to an institution. It is thereby possible to achieve a customized adjustment of the parameter values to be set and/or adjusted. User-specific boundary conditions shall be understood to mean boundary conditions that are specified by a user. Institution-specific boundary conditions shall be understood to mean boundary conditions that are specified in the same way for one institution, for instance in the same way for one hospital.

In another embodiment, the setting criteria include settings and/or adjustments of the parameter value of the at least one parameter of the magnetic resonance protocol that have already been made, and an evaluation of the already made settings and/or adjustments of the parameter value of the at least one parameter. The evaluation preferably involves information on how well and/or successful the adjusted and/or set parameter values comply with the previously specified boundary conditions. The evaluations have preferably been saved by a user. With this embodiment of the invention, it is possible to use the self-learning algorithm, in particular machine learning such as the deep-learning method, for instance, to optimize the setting and/or adjustment of the parameter values and hence continuously improve the acquired image data. In addition, it is possible to reduce user interactions during setting and/or adjustment of the parameter values, for example correcting the parameter values set and/or adjusted by the self-learning algorithm, in particular by machine learning such as the deep-learning method, for instance, and/or to dispense with these interactions completely.

In another embodiment of the invention, in the event of a transgression of the boundary conditions and/or of additional target specifications during the setting and/or adjustment of the parameter value of the at least one parameter of the magnetic resonance protocol, an alternative adjustment strategy is automatically selected in which a number of parameter values of additional parameters of the magnetic resonance protocol are adjusted and/or set. The additional target specifications may include safety specifications and/or patient-specific target specifications, etc. For example, an additional target specification may be a value for a specific absorption rate (SAR), which must not be exceeded during a magnetic resonance examination. The alternative adjustment strategy preferably involves adjusting and/or setting parameter values of more than one parameter, with the setting of parameter values of the number of parameters resulting in the boundary conditions and/or the additional target specifications being met. In addition, the alternative adjustment strategy can involve adjusting the boundary conditions.

It is thereby possible to adjust and/or correct the settable and/or adjustable parameter values and/or the boundary conditions automatically without the need to involve a user action. This allows the parameter values and/or boundary conditions to be corrected and/or adjusted particularly easily and quickly. The control computer, in particular the self-learning algorithm and/or machine learning such as the deep-learning method, for instance, of the control computer, preferably selects and implements the adjustment strategy.

Preferably, the adjustment strategy is selected for the specific situation. The control computer, in particular the self-learning algorithm and/or machine learning such as the deep-learning method, for instance, of the control computer, preferably performs the situation-specific selection of the adjustment strategy. This allows the individual parameters and/or boundary conditions to be corrected and/or adjusted to the additional target specifications in a manner that is particularly customized and specific to the current situation.

In another embodiment of the invention, the adjustment strategy adjusts and/or modifies those parameter values of the additional parameters of the magnetic resonance protocol that cause a minimum change in the boundary conditions and/or the setting criteria. This allows the individual parameters to be adjusted to the target specifications and to the boundary conditions and/or to the setting criteria in a particularly customized manner. In addition, the magnetic resonance sequence can be executed with only minimum deviations from an ideal setting.

In accordance with the invention, the set and/or adjusted parameter value of the at least one parameter of the magnetic resonance protocol is saved. In this case, the set and/or adjusted parameter value is preferably added to the database containing the already made settings and/or adjustments of the parameter value. It is thereby possible to continuously expand the available dataset containing already-made settings and/or adjustments of the parameter value and hence to minimize an adjustment risk and/or a setting risk for future settings and/or adjustments of the parameter value.

In another embodiment of the invention, the set and/or adjusted parameter value of the at least one parameter of the magnetic resonance protocol is evaluated, and the evaluation is saved together with the set and/or adjusted parameter value of the at least one parameter of the magnetic resonance protocol. The evaluation of the set and/or adjusted parameter value is preferably performed by a user via the user interface. Preferably, the evaluation is not performed until the magnetic resonance sequence has been executed. A user can use the evaluation to specify how well the set and/or adjusted parameter values meet the boundary conditions and/or the user's expectations. This can likewise be used to expand continuously the available dataset containing already made settings and/or adjustments of the parameter value, and hence to minimize an adjustment risk and/or a setting risk for future settings and/or adjustments of the parameter value. The evaluation can provide a dataset that allows recognition of patterns and regularities by means of the self-learning algorithm, in particular by means of machine learning such as the deep-learning method, for instance. It is thereby possible to use the self-learning algorithm to provide an optimized setting strategy and/or adjustment strategy for setting and/or adjusting the parameter values. In addition, the setting and/or adjusting of the parameter values can be adjusted in a customized and/or situation-specific manner by means of the self-learning algorithm.

Alternatively or additionally, the acquired image data can be saved in the database and/or the storage medium together with the set and/or adjusted parameter values. This makes it possible for the self-learning algorithm to recognize patterns and/or regularities in the acquired image data, and to derive therefrom a setting strategy and/or an adjustment strategy for setting and/or adjusting the parameter values.

The invention also concerns a magnetic resonance apparatus having a data acquisition scanner operated by a control computer and a user interface, wherein the magnetic resonance apparatus is designed to perform the method in accordance with the invention for setting and/or adjusting a parameter value of at least one parameter of a magnetic resonance protocol for at least one magnetic resonance sequence.

It is possible to achieve automated setting and/or adjustment of the parameter values for a magnetic resonance protocol and hence to provide simple and time-saving adjustment and/or setting of parameters for a user. All that is required of the user here for setting and/or adjusting the parameter values is just to select the magnetic resonance sequence and to specify and/or provide at least some of the boundary conditions. This allows new and/or inexperienced users to use individual magnetic resonance sequences optimally, with the result that the acquired magnetic resonance data can be of a higher quality. In addition, for the purpose of setting and/or adjusting the parameter value of the at least one parameter, all previously used and available setting criteria can be used automatically, thereby also reducing a possibility of mistakes when setting and/or adjusting the parameter values of the magnetic resonance protocol. This can also ensure that the acquired image data is of a high quality.

The advantages of the magnetic resonance apparatus according to the invention are essentially the same as the advantages detailed above of the method according to the invention for setting and/or adjusting a parameter value of at least one parameter of a magnetic resonance protocol for at least one magnetic resonance sequence. Features, advantages or alternative embodiments mentioned in connection with the method apply to the apparatus as well.

The present invention also encompasses a non-transitory, computer-readable data storage medium encoded with programming instructions (program code) that when the storage medium is loaded into a control computer of a magnetic resonance apparatus, causes the computer system to operate the magnetic resonance apparatus in order to implement any or all of the embodiments of the method according to the invention, as described above. The program code can be a source code, which still needs to be compiled and linked, or only needs to be interpreted, or may be executable software code that, for execution, needs only to be loaded into a suitable processor.

### BRIEF DESCRIPTION OF THE DRAWINGS

FIG. 1 schematically illustrates a magnetic resonance apparatus according to the invention.

FIG. 2 illustrates the a method according to the invention for setting and/or adjusting a parameter value of at least one parameter of a magnetic resonance protocol for at least one magnetic resonance sequence.

### DESCRIPTION OF THE PREFERRED EMBODIMENTS

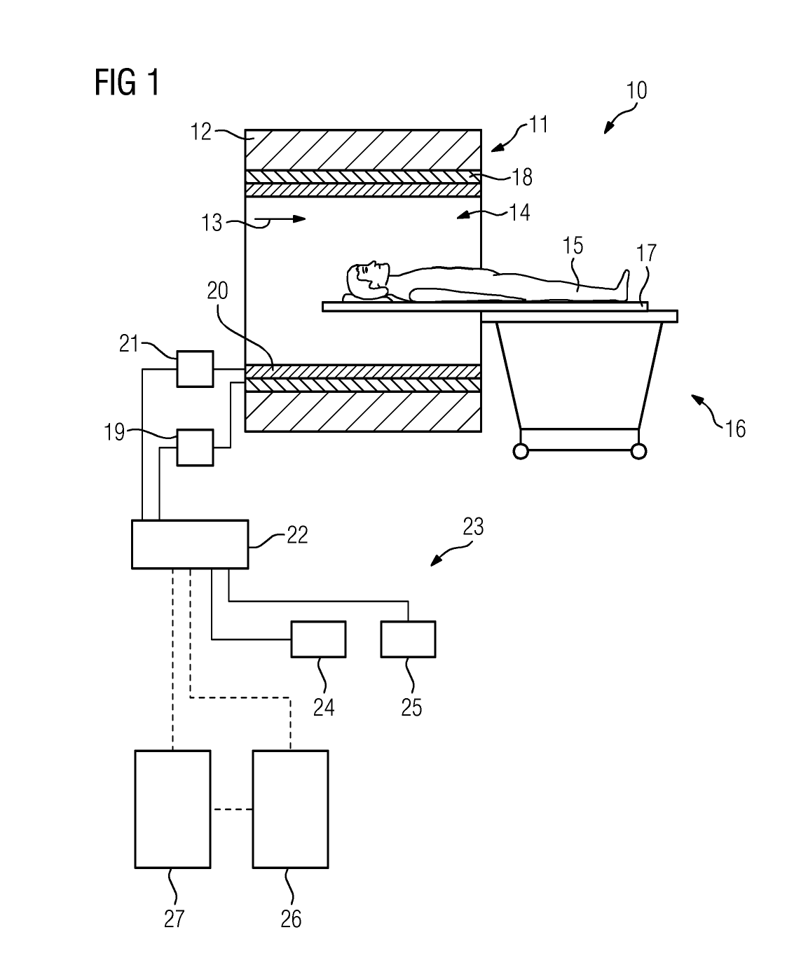

FIG. 1 schematically shows a magnetic resonance apparatus 10. The magnetic resonance apparatus 10 has a scanner 11, which contains a superconducting basic field magnet 12 that produces a strong and constant basic magnetic field 13. The scanner 11 has a patient receiving zone 14 for accommodating a patient 15. In the exemplary embodiment, the patient receiving zone 14 is shaped as a cylinder and is enclosed in a circumferential direction by the scanner 11. In principle, however, the patient receiving zone 14 can have a different design. The patient 15 can be moved into the patient receiving zone 14 by a patient support 16 of the scanner 11. The patient support apparatus 16 for this purpose has a patient table 17, which is designed to move inside the patient receiving zone 14.

The scanner 11 further has a gradient coil arrangement 18 for generating magnetic field gradients, which are used for spatially encoding the MR signals during imaging. The gradient coil arrangement 18 is controlled by a gradient controller 19 of the magnetic resonance apparatus 10. The scanner 11 further has an RF antenna 20 controlled by an RF antenna controller 21 so as to radiate an RF sequence into an examination space, which is largely formed by the patient receiving zone 14. The radiated RF sequence causes certain nuclear spins in the patient 15 to deviate from the field lines of the basic magnetic field 13, by an amount known as a flip angle. As the excited nuclear spins relax and thereby return to their steady state, they emit the aforementioned magnetic resonance signals. The magnetic resonance signals are detected by the same antenna that radiated a RF sequence, or by a different antenna.

The magnetic resonance apparatus 20 has a control computer 22 for controlling the basic field magnet 12, the gradient controller 18 and the RF antenna controller 20. The control computer 22 centrally controls the magnetic resonance apparatus 10, for instance implementing a predetermined imaging gradient echo sequence. In addition, the control computer 22 has an evaluation unit (not shown) for evaluating medical image data acquired during the magnetic resonance examination. In addition, the magnetic resonance apparatus 10 has a user interface 23, which is connected to the control computer 22. Control data such as imaging parameters, for instance, and reconstructed magnetic resonance images can be displayed to medical personnel on a display unit 24, for example on at least one monitor, of the user interface 23. The user interface also has an input unit 25.

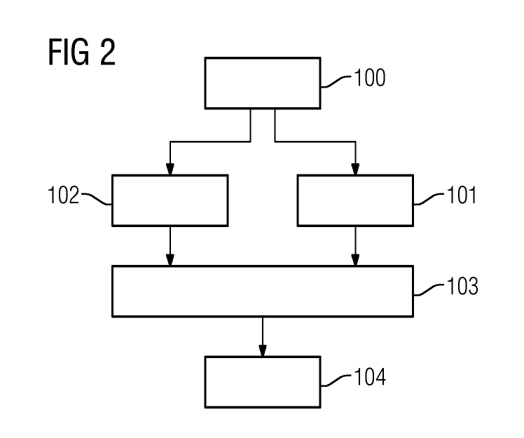

FIG. 2 shows a method according to the invention for setting and/or adjusting a parameter value of at least one parameter of a magnetic resonance protocol for at least one magnetic resonance sequence. The control computer 22 and a user interface 26 are available for performing the method for setting and/or adjusting a parameter value of at least one parameter of a magnetic resonance protocol for at least one magnetic resonance sequence.

The user interface 26 is embodied in this example as a user interface that is implemented separately from the magnetic resonance apparatus 10, the user interface 26 being embodied as a mobile user interface. In this case, the mobile user interface can be integrated in a mobile touch display and/or a tablet PC and/or a mobile phone etc. Alternatively or additionally, the user interface 23 of the magnetic resonance apparatus 10 can be available for performing the method for setting and/or adjusting a parameter value of at least one parameter of a magnetic resonance protocol for at least one magnetic resonance sequence.

For the purpose of performing the method for setting and/or adjusting a parameter value of at least one parameter of a magnetic resonance protocol for at least one magnetic resonance sequence, the control computer 22 has computer programs and/or software (code) that can be loaded directly into a memory unit 27 and comprise program means for performing a method for setting and/or adjusting measurement parameters for a measurement sequence of a magnetic resonance examination when the computer programs and/or software are executed in the control computer 22. The control computer 22 has for this purpose a processor (not shown), which is designed to execute the computer programs and/or software.

In the present exemplary embodiment, a memory that is outside the control computer 22 forms the unit 27. The cloud can serve as the memory 27. Alternatively, the computer programs and/or software can be stored in a memory 27 arranged inside the control computer 22.

In a first method step 100 of the method, the magnetic resonance protocol is selected. A user, for instance a member of medical operating staff supervising the magnetic resonance examination, can make the selection manually via the user interface 23 or 26. This selection can be made via the user interface 23 arranged inside the magnetic resonance apparatus 10 and via the user interface 26 implemented separately from the magnetic resonance apparatus 10. In addition, it is also possible for the selection to be made automatically and/or autonomously by the control computer 22 on the basis of patient information already present in the control computer 22.

In a subsequent method step 101, boundary conditions are provided for setting and/or adjusting the parameter value of the at least one parameter of the magnetic resonance protocol, wherein the user provides the boundary conditions by means of the user interface 23 or 26. Thus the boundary conditions are user-specific, in particular the user can specify the boundary conditions to achieve a particular measurement result. In addition, at least some of the boundary conditions can also be institution-specific in that at least individual boundary conditions are always the same every time a particular magnetic resonance sequence is used inside a hospital, for instance.

The boundary conditions for setting and/or adjusting the parameter value of the at least one parameter of the magnetic resonance protocol can here comprise a value range for the parameter value to be set and/or adjusted. The value range in this case defines a range within which the value of the set and/or adjusted parameter value must and/or should lie.

In addition, the boundary conditions for setting and/or adjusting the parameter value of the at least one parameter of the magnetic resonance protocol can also comprise an additional parameter value, wherein the additional parameter value involves a fixed parameter value. This fixed parameter value is preferably specified by the user. The fixed parameter value involves a constant value, which does not vary with adjustments and/or changes to additional parameter values, in particular when there is a change and/or adjustment to the parameter value to be set.

In addition, the boundary conditions for setting and/or adjusting the parameter value of the at least one parameter of the magnetic resonance protocol can also include target-oriented boundary conditions. The target-oriented boundary conditions can comprise, for example, a measurement time of the magnetic resonance sequence and/or an image quality of the acquired image data and/or a slice thickness and/or a contrast ratio and/or signal/noise ratio, etc. The above list does not constitute an exhaustive list here but only lists examples of the target-oriented boundary conditions.

For example, a user can define a T1 contrast measurement having a specified slice thickness and a defined image coverage as target-oriented boundary conditions for a magnetic resonance sequence.

In a further method step 102, setting criteria are provided from previous settings and/or adjustments of the parameter value of the at least one parameter of the magnetic resonance protocol. The method step 101 of providing boundary conditions for setting and/or adjusting the parameter value of the at least one parameter of the magnetic resonance protocol, and the method step 102 of providing setting criteria from previous settings and/or adjustments of the parameter value of the at least one parameter of the magnetic resonance protocol can be performed simultaneously or in any sequence.

The setting criteria can include settings and/or adjustments of the parameter value of the at least one parameter from earlier magnetic resonance examinations using this magnetic resonance sequence. In addition, the setting criteria can include evaluations for the previous settings and/or adjustments of the parameter value. The existing settings and/or adjustments of the parameter value and the evaluations of the settings and/or adjustments of the parameter value are stored in a storage medium and/or a database. In the present exemplary embodiment, the external memory 27 is or includes the storage medium and/or the database. In addition, in an alternative embodiment, it is also possible that the control computer 22 comprises the storage medium and/or the database. The control computer 22 can access the data stored in the external memory unit 27 by means of a data network.

Then in a further method step 103, after provision of the boundary conditions for setting and/or adjusting the parameter value of the at least one parameter of the magnetic resonance protocol, and after provision of the setting criteria from earlier settings and/or adjustments of the parameter value of the at least one parameter of the magnetic resonance protocol, the parameter value of the at least one parameter of the magnetic resonance protocol is set and/or adjusted. The parameter value is set and/or adjusted in this step on the basis of the provided boundary conditions and on the basis of the provided setting criteria.

The control computer 22 runs a self-learning algorithm in order to perform the method step of setting and/or adjusting the parameter value of the at least one parameter of the magnetic resonance protocol for the at least one magnetic resonance sequence. The parameter value of the at least one parameter is adjusted and/or set automatically and/or autonomously by the self-learning algorithm.

The self-learning algorithm is stored in this case inside the external memory 27. In addition, it is also possible for the self-learning algorithm to be stored inside a memory of the control computer 22.

The self-learning algorithm is based on machine learning, in particular on a deep-learning method, which involves generating knowledge from experience. In the deep-learning method, artificial neural networks are arranged in layers, which use ever more complex features to recognize the content of an image. It is hence possible, for example, to categorize large amounts of data.

The self-learning algorithm, in particular the deep-learning method, can hence recognize patterns and/or regularities in training data. The training data comprises the parameter values of the parameters, which values were previously set and/or adjusted and stored in the memory 27, and also the evaluation for these set and/or adjusted parameter values. In addition, the training data can also comprise the image data acquired using the set and/or adjusted parameter values of the magnetic resonance sequence, so that additional criteria, for instance criteria such as an image contrast etc., are available for recognizing patterns and/or regularities in the training data.

The data already available on the set and/or adjusted parameter values is used here to train the self-learning algorithm, in particular machine learning such as the deep-learning method, for instance, with regard to the boundary conditions. For example, the self-learning algorithm can be trained with regard to an image contrast and/or a T1 contrast and/or other boundary conditions deemed useful by those skilled in the art.

For example, it is sufficient for setting and/or adjusting parameter values if the user defines the boundary conditions, for instance a magnetic resonance examination having a T1 contrast and a specified slice thickness and image coverage. All further parameter values to be set and/or adjusted of parameters of the magnetic resonance protocol are subsequently determined and set automatically and/or autonomously by the self-learning algorithm, taking into account the defined and/or provided boundary conditions and the defined and/or provided setting criteria.

The method step of setting and/or adjusting the parameter value of the at least one parameter of the magnetic resonance protocol in addition checks and/or examines whether additional target specifications are met. The additional target specifications may comprise safety specifications and/or patient-specific target specifications such as a specific absorption rate, for instance.

If it is established in the method step of setting and/or adjusting the parameter value of the at least one parameter of the magnetic resonance protocol that setting and/or adjusting the parameter value in accordance with the boundary conditions specifications and in accordance with the setting criteria specifications would result in a transgression of an additional target specification, the self-learning algorithm selects an alternative adjustment strategy. The alternative adjustment strategy involves modifying and/or adjusting a number of parameter values of additional parameters of the magnetic resonance protocol. In addition, the alternative adjustment strategy can also result in a change to boundary conditions.

The self-learning algorithm selects said alternative adjustment strategy for the specific situation. In addition, parameter values of those parameters of the magnetic resonance protocol that cause a minimum change in the boundary conditions and/or the setting criteria are adjusted and/or modified.

Once the parameter values have been set and/or adjusted, these parameter values are saved and are hence available for a subsequent use of the magnetic resonance sequence. In this case, the parameter values are saved in the storage medium and/or the database containing the already preceding settings and/or adjustments of parameter values. In addition, after the magnetic resonance sequence has been executed, a user also issues an evaluation via the user interface, and the evaluation is saved together with the adjusted and/or set parameter values. The set and/or adjusted parameter values together with the evaluation for the adjustment and/or setting of the parameter values are saved in a further method step 104. In the method step 104, the image data acquired using the magnetic resonance sequence can additionally also be saved together with the set and/or adjusted parameter values.

The method presented above is described with reference to setting and/or adjusting one parameter value of one parameter of the magnetic resonance protocol as an example. In principle, the method can be performed for any number of parameter values and/or any number of parameters of the magnetic resonance protocol without departing from the scope of the invention.

Although modifications and changes may be suggested by those skilled in the art, it is the intention of the Applicant to embody within the patent warranted hereon all changes and modifications as reasonably and properly come within the scope of the Applicant's contribution to the art.

1. A method for setting and/or adjusting a parameter value of a parameter of a magnetic resonance (MR) protocol for an MR sequence, said method comprising:
in a computer, receiving a manual entry that designates a selection of an MR protocol in order to operate an MR apparatus;
also in said computer, receiving a manual entry that designates boundary conditions for setting and/or adjusting a parameter value of a parameter of the selected MR protocol;
in said computer, generating setting criteria from previous settings and/or adjustments of said parameter value of said parameter of said MR protocol;
in said computer, setting and/or adjusting said parameter value of said parameter of said MR protocol based on the designated boundary conditions and the generated setting criteria; and
making the set and/or adjusted parameter value of said parameter of said MR protocol available in electronic from said computer.

2. A method as claimed in claim 1 comprising setting and/or adjusting said parameter value of said parameter of said MR protocol in said computer automatically by executing a self-learning algorithm.

3. A method as claimed in claim 2 comprising executing said self-learning algorithm so as to use all previous settings and/or adjustments of said parameter value of said parameter of said MR protocol, accessed from a memory by said computer.

4. A method as claimed in claim 1 comprising, in said boundary conditions, designating a value range for the parameter value of said parameter of said MR protocol.

5. A method as claimed in claim 1 comprising, in said boundary conditions, designating at least one additional parameter value of an additional parameter of said MR protocol, said additional parameter value being a fixed parameter value.

6. A method as claimed in claim 1 comprising designating said boundary conditions so as to include target-oriented boundary conditions.

7. A method as claimed in claim 1 comprising selecting said target-oriented boundary conditions from the group consisting of a measurement time for execution of said MR protocol, an image quality of an MR image obtained with said MR protocol, a slice thickness from which MR data are to be obtained by execution of said MR protocol, a contrast ratio in an image produced from MR data acquired by execution of said MR protocol, and a signal-to-noise ratio of MR signals acquired by execution of said MR protocol.

8. A method as claimed in claim 1 comprising making said boundary conditions specific to an operator who makes said manual entry of said boundary conditions.

9. A method as claimed in claim 1 comprising generating said setting criteria so as to comprise settings and/or adjustments of said parameter value of said parameter of said MR protocol that have previously been made by evaluating said previously made settings and/or adjustments.

10. A method as claimed in claim 1 comprising if said boundary conditions are transgressed, automatically selecting an alternative adjustment strategy in said computer in which a plurality of parameter values of additional parameters of said MR protocol are adjusted and/or set.

11. A method as claimed in claim 10 comprising selecting said adjustment strategy dependent on a predetermined requirement.

12. A method as claimed in claim 10 comprising selecting said alternative adjustment strategy so as to cause a minimum change in said boundary conditions and/or said setting criteria.

13. A method as claimed in claim 1 comprising saving, in a memory, the set and/or adjusted parameter value of said parameter of said MR protocol.

14. A method as claimed in claim 13 comprising, in said computer, evaluating said set and/or adjusted parameter value of said parameter of said MR protocol in order to obtain an evaluation result, and saving said evaluation result in said memory together with the set and/or adjusted parameter value of said parameter of said MR protocol in said memory.

15. A magnetic resonance (MR) apparatus comprising:
an MR data acquisition scanner;
a control computer configured to operate said MR data acquisition scanner according to an MR protocol, said computer comprising a user interface;
said computer being configured to receive, vis said user interface, a manual entry that designates a selection of an MR protocol in order to operate an MR apparatus as a selected protocol;
said computer being configured to receive, via said manual interface, a manual entry that designates boundary conditions for setting and/or adjusting a parameter value of a parameter of the selected MR protocol;
said computer being configured to generate setting criteria from previous settings and/or adjustments of said parameter value of said parameter of said MR protocol;
said computer being configured to set and/or adjust said parameter value of said parameter of said MR protocol based on the designated boundary conditions and the generated setting criteria; and
said computer being configured to make the set and/or adjusted parameter value of said parameter of said MR protocol available in electronic from said computer.

16. A non-transitory, computer-readable data storage medium encoded with programming instructions, said storage medium being loaded into a control computer of a magnetic resonance (MR) apparatus, and said programming instructions causing said control computer to:
receive a manual entry that designates a selection of an MR protocol in order to operate an MR apparatus;
receive a manual entry that designates boundary conditions for setting and/or adjusting a parameter value of a parameter of the selected MR protocol;
generate setting criteria from previous settings and/or adjustments of said parameter value of said parameter of said MR protocol;
set and/or adjust said parameter value of said parameter of said MR protocol based on the designated boundary conditions and the generated setting criteria; and
make the set and/or adjusted parameter value of said parameter of said MR protocol available in electronic from said computer.
